(19) United States
(12) Patent Application Publication
(10) Pub. No.: US 2019/0271752 A1
(43) Pub. Date: Sep. 5, 2019

(54) MAGNETIC RESONANCE ANGIOGRAPHY

(71) Applicant: Shenyang Neusoft Medical Systems Co., Ltd., Shenyang (CN)

(72) Inventors: Wei XU, Shenyang (CN); Cao CHEN, Shenyang (CN); Tiecheng LI, Shenyang (CN)

(21) Appl. No.: 16/291,749

(22) Filed: Mar. 4, 2019

(30) Foreign Application Priority Data
Mar. 2, 2018 (CN) 201810175899.9

(51) Int. Cl. G01R 33/563; G01R 33/561; A61B 6/00

(52) U.S. Cl. CPC G01R 33/5635; A61B 6/504; G01R 33/5615

(57) ABSTRACT

A magnetic resonance angiography method includes: in a plurality of first repeated collecting periods, a first echo signal set is formed by flow-compensated first echo signals and a second echo signal set is formed by flow-compensated second echo signals; in a plurality of second repeated collecting periods, a third echo signal set is formed by flow-compensated third echo signals and a fourth echo signal set is formed by flow-dephased fourth echo signals; a venous blood vessel image is reconstructed according to the second echo signal set; an arteriovenous blood vessel image is obtained according to the first echo signal set, the third echo signal set and the fourth echo signal set; and an arterial blood vessel image is obtained according to the venous blood vessel image and the arteriovenous blood vessel image.

### CROSS REFERENCE TO RELATED APPLICATIONS

This application claims priority to Chinese Patent Application No. 201810175899.9 and filed on Mar. 2, 2018, the entire content of which is incorporated herein by reference.

### TECHNICAL FIELD

The present disclosure relates to magnetic resonance angiography in the field of medical technology.

### BACKGROUND

Magnetic Resonance Imaging (MRI) is an imaging method of generating a detectable signal through interaction between a radiofrequency wave and a nucleus system in an external magnetic field. The essence of the MRI is a quantum effect for a transition between energy levels. A principle of the MRI is to place a subject (e.g., a patient) in a magnetic field environment and excite hydrogen nucleuses in the subject with radiofrequency pulses, so as to trigger that the hydrogen nucleuses resonate and absorb energy. After the radiofrequency pulses are stopped, the hydrogen nucleuses emit radiofrequency signals at a particular frequency and release the previously-absorbed energy. An external detecting apparatus receives the radiofrequency signals released by the subject, converts the radiofrequency signals into image signals, and generates an image by using the image signals. The MRI is widely used as an effective examination method in diagnoses of clinical diseases because the features of the MRI, such as without ionizing radiation, multiple collected parameters, a relatively large amount of information, multi-directional imaging, a relatively high resolution for soft tissues and so on.

Magnetic Resonance Angiography (MRA) is a typical application of the MRI technology, which is a non-invasive angiography method without a cannula and a contrast agent.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS' products are exported to over 60 countries and regions around the globe, serving more than 5,000 renowned customers. NMS's latest successful developments, such as 128. Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during the CT scanning process.

### SUMMARY

The present disclosure provides methods, devices, and systems for magnetic resonance angiography in the field of medical technology.

In general, one innovative aspect of the subject matter described in this specification can be embodied in methods that include the actions for, including: forming a first echo signal set in a plurality of first repeated collecting periods by collecting a respective first echo signal within a first time interval included in each of the first repeated collecting periods, the respective first echo signals being flow-compensated; forming a second echo signal set in the plurality of first repeated collecting periods by collecting a respective second echo signal within a second time interval included in each of the first repeated collecting periods, the respective second echo signals being flow-compensated; forming a third echo signal set in a plurality of second repeated collecting periods by obtaining third echo signals within third time intervals included in the plurality of second repeated collecting periods, the third echo signals being flow-compensated; forming a fourth echo signal set in the plurality of second repeated collecting periods by obtaining fourth echo signals within fourth time intervals included in the plurality of second repeated collecting periods, the fourth echo signals being flow-dephased; reconstructing a venous blood vessel image according to the second echo signal set; obtaining an arteriovenous blood vessel image according to the first echo signal set, the third echo signal set and the fourth echo signal set; and obtaining an arterial blood vessel image according to the venous blood vessel image and the arteriovenous blood vessel image.

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. For example, a first echo time for the first time interval can be the same as a third echo time for the third time interval, a second echo time for the second time interval can be the same as a fourth echo time for the fourth time interval, and the second echo time can be longer than the first echo time, and the fourth echo time can be longer than the third echo time.

In some implementations, forming a third echo signal set in a plurality of second repeated collecting periods includes: collecting a respective third echo signal within a third time interval included in each of the second repeated collecting periods.

In some implementations, forming a third echo signal set in a plurality of second repeated collecting periods includes: dividing a third K-space corresponding to the third time intervals into a first sub-space and a second sub-space, wherein an absolute value of a difference between an index of each of phase encoding lines in the first sub-space and an index of a central phase encoding line in the third K-space is less than a first threshold, and an absolute value of a difference between an index of each of phase encoding lines in the second sub-space and the index of the central phase encoding line in the third K-space is greater than or equal to the first threshold; collecting a first sub-space echo signal within the first sub-space in the third time interval included in each of the second repeated collecting periods to form a first sub-space echo signal set, wherein the first sub-space echo signals have been flow-compensated; selecting a second sub-space echo signal set corresponding to the second sub-space from the first echo signal set; and forming the third echo signal set by combining the first sub-space echo signal set and the second sub-space echo signal set.

In some examples, selecting the second sub-space echo signal set corresponding to the second sub-space from the first echo signal set includes: dividing a first K-space corresponding to the first time intervals into a third sub-space and a fourth sub-space, where the first echo signals of the first echo signal set are filled in rows of the first K-space, and where an absolute value of a difference between an index of each of phase encoding lines in the third sub-space and an index of a central phase encoding line in the first K-space is less than the first threshold, and an absolute of a difference between an index of each of phase encoding lines in the fourth sub-space and the index of the central phase encoding line in the first K-space is greater than or equal to the first threshold; and selecting a fourth sub-space echo signal set corresponding to the fourth sub-space from the first echo signal set as the second sub-space echo signal set corresponding to the second sub-space.

In some implementations, forming a fourth echo signal set in the plurality of second repeated collecting periods includes: collecting a respective fourth echo signal within a fourth time interval included in each of the second repeated collecting periods.

In some implementations, forming a fourth echo signal set in the plurality of second repeated collecting periods includes: dividing a fourth K-space corresponding to the fourth time interval into a fifth sub-space and a sixth sub-space, wherein an absolute of a difference between an index of each of phase encoding lines in the fifth sub-space and an index of a central phase encoding line in the fourth K-space is less than a second threshold, and an absolute of a difference between an index of each of phase encoding lines in the sixth sub-space and the index of the central phase encoding line in the fourth K-space is greater than or equal to the second threshold; collecting a fifth sub-space echo signal within the fifth sub-space in each of the second repeated collecting periods to form a fifth sub-space echo signal set, wherein the fifth sub-space echo signals have been flow-dephased; obtaining a sixth sub-space echo signal set with a zero filling strategy; and forming the fourth echo signal set by combining the fifth sub-space echo signal set and the sixth sub-space echo signal set. Obtaining the sixth sub-space echo signal set with the zero filling strategy can include: filling each of sixth sub-space echo signals in the sixth sub-space echo signal set with 0.

In some implementations, obtaining the arteriovenous blood vessel image according to the first echo signal set, the third echo signal set and the fourth echo signal set includes: obtaining a first sub-image according to the first echo signal set; obtaining a second sub-image according to the third echo signal set; obtaining a third sub-image according to at least one of the first sub-image and the second sub-image; obtaining a fourth sub-image according to the fourth echo signal set; and obtaining the arteriovenous blood vessel image according to the third sub-image and the fourth sub-image.

Obtaining the third sub-image according to at least one of the first sub-image and the second sub-image can include one of the following: determining the first sub-image as the third sub-image; determining the second sub-image as the third sub-image; and obtaining the third sub-image by performing averaging processing on the first sub-image and the second sub-image.

Obtaining the arteriovenous blood vessel image according to the third sub-image and the fourth sub-image can include: obtaining the arteriovenous blood vessel image by performing subtracting processing on the third sub-image and the fourth sub-image.

Obtaining the arterial blood vessel image according to the venous blood vessel image and the arteriovenous blood vessel image can include: obtaining the arterial blood vessel image by performing subtracting processing on the arteriovenous blood vessel image and the venous blood vessel image.

Another aspect of the present disclosure features a magnetic resonance angiography method, including: in each of a plurality of first repeated collecting periods, collecting a first echo signal within a first time interval included in the first repeated collecting period to form a first echo signal set, where the first echo signal has been flow-compensated and the first echo signal set includes the first echo signals collected in the plurality of first repeated collecting periods, and collecting a second echo signal within a second time interval included in the first repeated collecting period to form a second echo signal set, where the second echo signal has been flow-compensated and the second echo signal set includes the second echo signals collected in the plurality of first repeated collecting periods; in each of a plurality of second repeated collecting periods, obtaining a third echo signal within a third time interval included in the second repeated collecting period to form a third echo signal set, where the third echo signal has been flow-compensated and the third echo signal set includes the third echo signals obtained in the plurality of second repeated collecting periods, and obtaining a fourth echo signal within a fourth time interval included in the second collecting period to form a fourth echo signal set, where the fourth echo signal has been flow-dephased and the fourth echo signal set includes the fourth echo signals obtained in the plurality of second repeated collecting periods; reconstructing a venous blood vessel image according to the second echo signal set; obtaining an arteriovenous blood vessel image according to the first echo signal set, the third echo signal set and the fourth echo signal set; and obtaining an arterial blood vessel image according to the venous blood vessel image and the arteriovenous blood vessel image.

A number of the plurality of second repeated collecting periods can be identical to a number of the plurality of first repeated collecting periods. Each of the plurality of second repeated collecting periods can be sequential to a corresponding first repeated collecting period.

The details of one or more examples of the subject matter described in the present disclosure are set forth in the accompanying drawings and description below. Other features, aspects, and advantages of the subject matter will become apparent from the description, the drawings, and the claims. Features of the present disclosure are illustrated by way of example and not limited in the following figures, in which like numerals indicate like elements.

### BRIEF DESCRIPTION OF DRAWINGS

FIG. 1 is a schematic diagram illustrating a cross section of an MRI device according to one or more examples of the present disclosure.

FIG. 2 is a schematic diagram illustrating a spatial encoding manner according to one or more examples of the present disclosure.

FIG. 3 is a flowchart illustrating a magnetic resonance angiography method according to one or more examples of the present disclosure.

FIG. 4A is a schematic diagram illustrating a K-space according to one or more examples of the present disclosure.

FIG. 4B is a schematic diagram illustrating the division of the K-space according to one or more examples of the present disclosure.

FIG. 5 is a schematic diagram illustrating a hardware structure of a magnetic resonance angiography apparatus according to one or more examples of the present disclosure.

FIG. 6 is a schematic diagram illustrating a structure of a magnetic resonance angiography logic according to one or more examples of the present disclosure.

### DETAILED DESCRIPTION

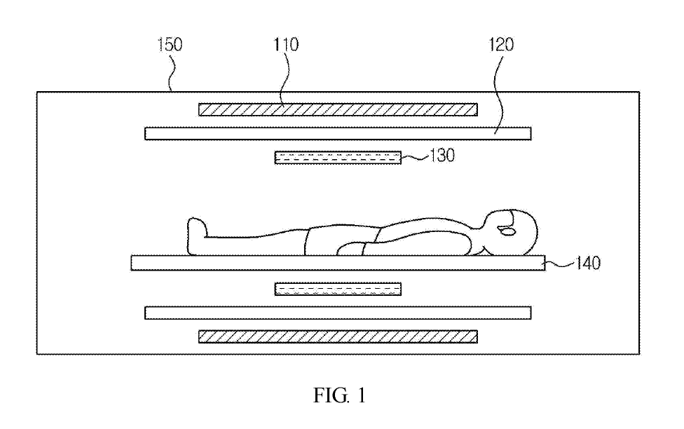

A magnetic resonance angiography method is provided in examples of the present disclosure, which can be applied to an MRI device. FIG. 1 is a schematic diagram illustrating a cross section of an MRI device according to one or more examples of the present disclosure. The MRI device may include a main magnetic field 150, a gradient coil 110, a radiofrequency transmitting coil 120 and a radiofrequency receiving coil 130. Certainly, the MRI device may also include other components, which is not limited herein. Descriptions are made with the structure shown in FIG. 1 as an example.

The main magnetic field 150 is configured to provide a magnetic field environment for imaging. After a subject is placed on a scanning bed 140, a corresponding scanning region of the subject is located within the magnetic field environment provided by the main magnetic field 150. The gradient coil 110, the radio frequency transmitting coil 120 and the radiofrequency receiving coil 130 are located within the magnetic field environment provided by the main magnetic field 150.

The radiofrequency transmitting coil 120 is configured to transmit a pulse signal at a designated scanning position, such as the head of the subject, the heart of the subject and so on). The pulse signal is used to excite hydrogen nucleuses within the subject and cause the resonance of the hydrogen nucleuses. After receiving the pulse signal, the subject emits a radiofrequency signal at a particular frequency, where the radiofrequency signal is a resonance signal of the above pulse signal, and the radiofrequency signal generated by the subject is received by the radiofrequency receiving coil 130.

After receiving the radiofrequency signal, the radiofrequency receiving coil 130 is configured to transmit the radiofrequency signal to a spectrometer. The spectrometer is configured to analyze the radiofrequency signal, convert the radiofrequency signal into an image signal, and then transmit the image signal to a computer. The computer is configured to generate an image by using the image signal and provide the image to a medical staff for diagnosis.

When the radiofrequency transmitting coil 120 transmits the pulse signal, the gradient coil 110 is configured to provide spatial encoding information to the subject, so that the excited region of the subject generates the radiofrequency signal based on the spatial encoding information under the excitation of the pulse signal. That is, the radiofrequency signal may include the spatial encoding information. The radiofrequency signal received by the radiofrequency receiving coil 130 includes the spatial encoding information. Tissue information of different regions is finally reconstructed from a reconstructed image. For example, the computer may generate the reconstructed image by using the image signal including the spatial encoding information.

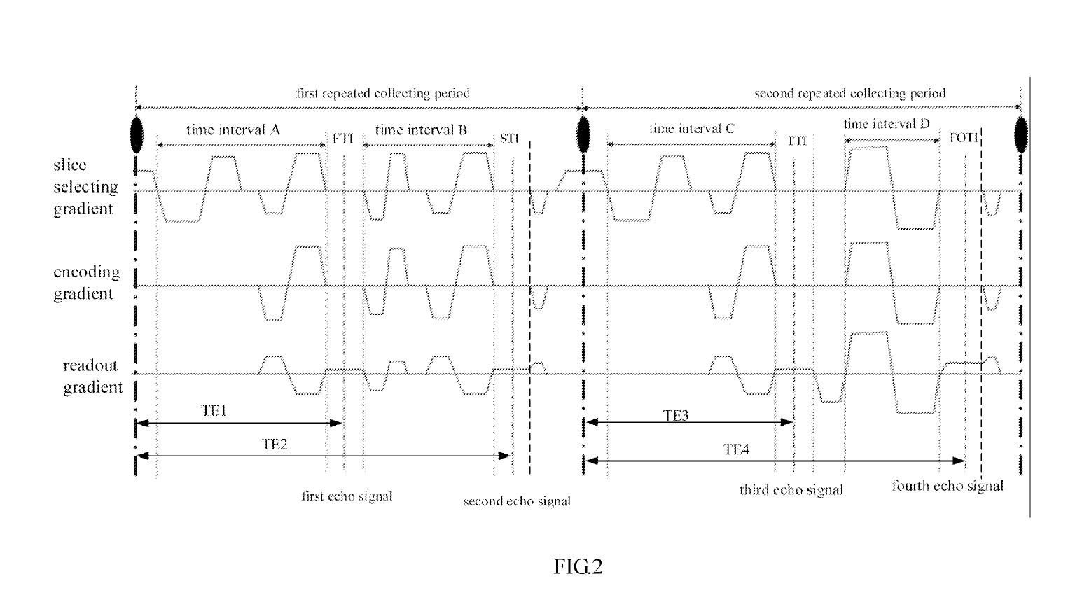

In one or more examples of the present disclosure, to obtain a venous blood vessel image (e.g., an image of a cerebral venous blood vessel) and an arterial blood vessel image (e.g., an image of a cerebral arterial blood vessel), a spatial encoding manner shown in FIG. 2 may also be adopted. Certainly, the spatial encoding manner shown in FIG. 2 is only an example, which is based on a gradient echo. The spatial encoding manner shown in FIG. 2 will be described below as an example.

As shown in FIG. 2, each collecting period may be divided into a first repeated collecting period and a second repeated collecting period. Further, a time length of the first repeated collecting period is the same as a time length of the second repeated collecting period. For example, the time length of the first repeated collecting period and the time length of the second repeated collecting period are both 50 milliseconds.

As shown in FIG. 2, the first repeated collecting period may include: a first time interval (FTI), a second time interval (STI), a time interval A, a time interval B, and the like. In addition, the second repeated collecting period may include: a third time interval (TTI), a fourth time interval (FOTI), a time interval C, a time interval D, and the like.

In an example of FIG. 2, the radiofrequency receiving coil 130 is configured to receive a first echo signal in the first time interval (a collecting time length of the first echo signal) and a second echo signal in the second time interval (a collecting time length of the second echo signal) within each first repeated collecting period, and a third echo signal in the third time interval (a collecting time length of the third echo signal) and a fourth echo signal in the fourth time interval (a collecting time length of the fourth echo signal) within each second repeated collecting period.

As shown in FIG. 2, take one collecting period as an example. When the gradient coil 110 provides first spatial encoding information to the subject during the time interval A, the time interval B and the time interval C, the first spatial encoding information is used to perform flow compensation for a flowing tissue (e.g., blood) of the subject, so that an echo signal generated by the subject is a flow-compensated echo signal. When the gradient coil 110 provides second spatial encoding information to the subject during the time interval D, the spatial encoding information is used to perform flow dephasing (may also referred to as phase divergence or active phase divergence) for the flowing tissue of the subject, so that an echo signal generated by the subject is a flow-dephased echo signal.

The gradient coil 110 may provide the first spatial encoding information for flow compensation or the second spatial encoding information for flow dephasing to the subject based on different shapes of a slice selecting gradient, an encoding gradient and a readout gradient. By setting gradient coil 110, the different shapes of the slice selecting gradient, the encoding gradient and the readout gradient are provided. For example, the gradient coil 110 may provide the first spatial encoding information for flow compensation based on the slice selecting gradient, the encoding gradient and the readout gradient within the time interval A; the gradient coil 110 may provide the first spatial encoding information for flow compensation based on the slice selecting gradient, the encoding gradient and the readout gradient within the time interval B; the gradient coil 110 may provide the first spatial encoding information for flow compensation based on the slice selecting gradient, the encoding gradient and the readout gradient within the time interval C, where the slice selecting gradient, the encoding gradient and the readout gradient within the time interval C may respectively be the same as those within the time interval A, which will not be described herein; and the gradient coil 110 may also provide the second spatial encoding information for flow dephasing based on the slice selecting gradient, the encoding gradient and the readout gradient within the time interval D. Certainly, each of the slice selecting gradient, the encoding gradient, and the readout gradient shown in FIG. 2 is only an example used to implement the function of the flow compensation or the flow dephasing, which is not limited herein.

In conclusion, the flow compensation may be performed on the flowing tissue of the subject by providing the slice selecting gradient, the encoding gradient and the readout gradient shown in FIG. 2 to the subject during the time interval A, the time interval B and the time interval C. In this way, the subject may emit a flow-compensated echo signal. Similarly, the subject may emit a flow-dephased echo signal during the time interval D.

For example, if the flowing tissue is blood, performing flow compensation on the blood of the subject refers to: because the blood is in a flowing state, the flow of the blood weakens a signal of an imaging pixel (may also be referred to as a blood tissue signal), and it is considered that the blood flows at an approximately constant speed, a blood tissue signal of a blood vessel in which the blood flows at a constant speed is retained with the flow compensation, thereby avoiding weakening the blood tissue signal. Thus, the flow-compensated echo signal may include all blood tissue signals, including signals lost due to blood flow. In addition, performing flow dephasing on the blood of the subject refers to: because it is considered that the blood flows at a constant speed, a blood tissue signal corresponding to a particular flow speed may be diverged with an encoding gradient corresponding to the flow speed, thereby attenuating the blood tissue signal. Therefore, the flow-dephased echo signal may include attenuated blood tissue signals. The slice selecting gradient, the encoding gradient and the readout gradient corresponding to the fourth echo signal are not designed with the flow compensation, but added with a flow-dephased bipolar gradient. In this case, with the additional gradient phase divergence, the signal of the flowing tissue such as a blood tissue is diverged faster, that is, the collected flowing tissue signal is relatively weak.

In the above examples, flow compensation or flow dephasing may be performed on the flowing tissue of the subject with the slice selecting gradient, the encoding gradient and the readout gradient. Certainly, the slice selecting gradient, the encoding gradient and the readout gradient also have other functions, which is not limited herein. In addition, the slice selecting gradient, the encoding gradient and the readout gradient shown in FIG. 2 are only examples for implementing flow compensation or flow dephasing, which are not limited herein.

Echo time (TE) is a time period between an excitation pulse and a peak of an echo signal of the excitation pulse. In the above examples, a first echo time (TE1) for the first time interval remains the same as a third echo time (TE3) for the third time interval. A second echo time (TE2) for the second time interval remains the same as a fourth echo time (TE4) for the fourth time interval. The second echo time is longer than the first echo time and the fourth echo time is longer than the third echo time.

For example, the first echo time is shorter, such as 10 milliseconds, and the third echo time is the same as the first echo time. The second echo time is longer, such as 40 milliseconds, and the fourth echo time is the same as the second echo time.

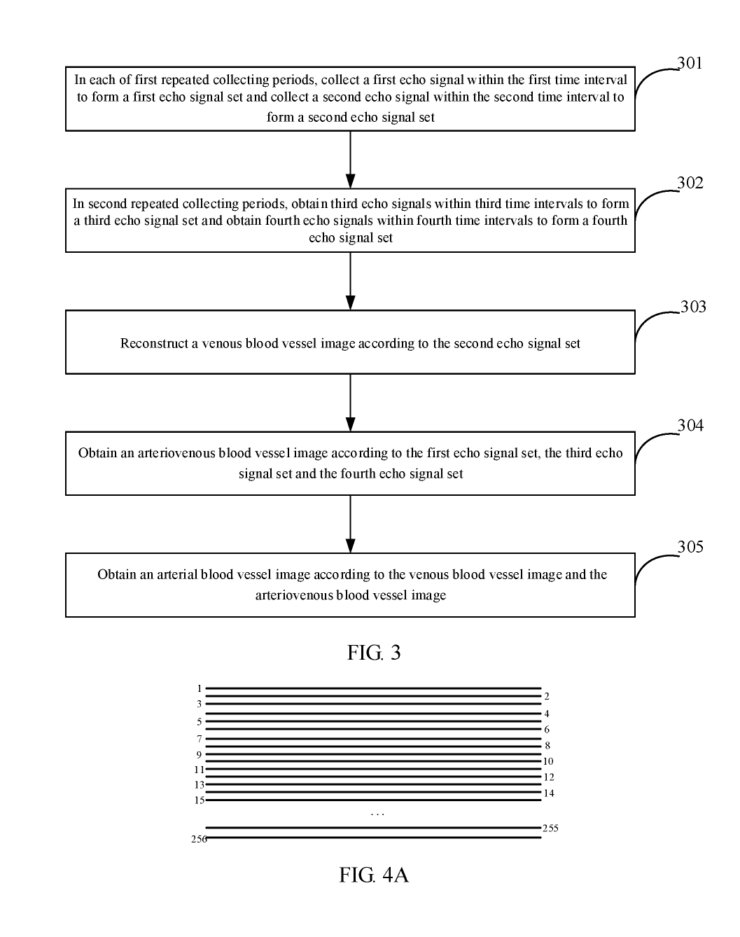

In the above application scenario, FIG. 3 is a flowchart illustrating a magnetic resonance angiography method according to one or more examples of the present disclosure. As shown in FIG. 3, the method may be applied to a medical device (e.g., an MRI device), and include the following steps.

At step 301, in each of first repeated collecting periods, a first echo signal within the first time interval is collected to form a first echo signal set, and a second echo signal within the second time interval is collected to form a second echo signal set. It is noted that the first echo signal ant the second echo signal both have been flow-compensated.

In each of the first repeated collecting periods, collecting the first echo signal within the first time interval to form the first echo signal set can include: in each of the first repeated collecting periods, collecting the first echo signals within a first K-space corresponding to the first time interval to form the first echo signal set. The first echo signal set includes the first echo signal collected within each of the first repeated collecting periods, and each of the first echo signals corresponds to a respective spatial phase code.

As shown in FIG. 2, the gradient coil 110 is configured to provide spatial encoding information to the subject, where the spatial encoding information is used to perform flow compensation on the flowing tissue of the subject. In the first time interval of a first repeated collecting period of the first repeated collecting periods, the radiofrequency receiving coil 130 receives a first echo signal generated by the subject as a first one of the first echo signal set, where the first one of the first echo signal set is used to fill a first row in the first K-space. In the first time interval of a second first repeated collecting period of the first repeated collecting periods, by adjusting the spatial phase encoding, the radiofrequency receiving coil 130 receives a first echo signal generated by the subject as a second one of the first echo signal set, where the second one of the first echo signal set is used to fill a second row in the first K-space, and so on. In the first time interval of an N-th first repeated collecting period of the first repeated collecting periods, by adjusting the spatial phase encoding, the radiofrequency receiving coil 130 receives a first echo signal generated by the subject as an N-th one of the first echo signal set, where the N-th one of the first echo signal set is used to fill an N-th row in the first K-space. In the N number of the first repeated collecting periods, the first echo signal set includes the N number of first echo signals. N is an integer greater than 1.

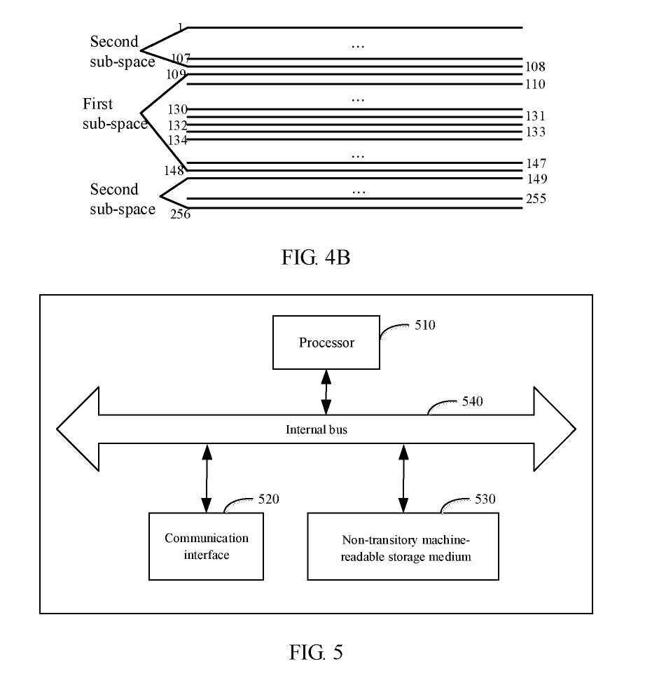

As shown in FIG. 4A, if the first K-space corresponding to the first time interval includes 256 rows, the first echo signal set includes 256 first echo signals. After the first of the first echo signal set is collected, the spatial phase encoding is adjusted in each first repeated collecting period. The adjustment process is not limited herein. The 256 first echo signals are finally collected and these first echo signals form the first echo signal set.

In FIG. 4A, each straight line indicates one first echo signal, and there are totally 256 rows. Certainly, the above indication is only an example, and is not limited to 256 echo signals in an actual collecting process.

In each of the first repeated collecting periods, collecting the second echo signal within the second time interval to form the second echo signal set includes: in each of the first repeated collecting periods, collecting the second echo signals within a second K-space corresponding to the second time interval to form the second echo signal set. The second echo signal set includes the second echo signal collected within each of the first repeated collecting periods, and each of the second echo signals corresponds to a respective spatial phase code.

The second echo signal set may be obtained in a manner similar to that of the first echo signal set, which is not described herein.

At step 302, in second repeated collecting periods, third echo signals within the third time intervals are obtained to form a third echo signal set, and fourth echo signals within the fourth time intervals are obtained to form a fourth echo signal set. It is noted that the third echo signals have been flow-compensated and the fourth echo signal have been flow-dephased.

In some cases, obtaining the third echo signals within the third time intervals in the second repeated collecting periods to form the third echo signal set may be performed in any one of a first manner and a second manner.

In the first manner, the third echo signal within a third K-space corresponding to the third time interval in each of a plurality of second repeated collecting periods is collected to form the third echo signal set. A number of the plurality of second repeated collecting periods is identical to a number of the plurality of first repeated collecting periods. Each of the second repeated collecting periods is sequential to a corresponding first repeated collecting period. The third echo signal set includes the third echo signals collected within the second repeated collecting periods, and each of the third echo signals corresponds to a respective spatial phase code. The third echo signal set may be obtained in a manner similar to that of the first echo signal set, which is not described herein.

In the second manner, the third K-space corresponding to the third time interval is divided into a first sub-space and a second sub-space, both of which form the third K-space corresponding to the third time interval; a first sub-space echo signal within the first sub-space in each of the second repeated collecting periods is collected to form a first sub-space echo signal set, without collecting a flow-compensated second sub-space echo signal within the second sub-space in each of the second repeated collecting periods; a second sub-space echo signal set corresponding to the second sub-space is selected from the first echo signal set corresponding to the first K-space; and the third echo signal set is formed by combining the first sub-space echo signal set with the second sub-space echo signal set. Each of the first sub-space echo signals has been flow-compensated with the spatial encoding information provided by the gradient coil 110.

When the third K-space corresponding to the third time interval is divided into the first sub-space and the second sub-space, an absolute value of a difference between an index of each of phase encoding lines in the first sub-space and an index of a central phase encoding line in the third K-space is less than a first threshold (which may be configured to be, for example, 20 based on experiences), an absolute value of a difference between an index of each of phase encoding lines in the second sub-space and the index of the central phase encoding line in the third K-space is greater than or equal to the first threshold, and the first sub-space and the second sub-space can be completely equivalent to the third K-space.

For example, if the third K-space corresponding to the third time interval includes 256 rows, that is, 256 phase encoding lines and the first threshold is 20, the index of the central phase encoding line in the third K-space refer to 128.5, the first sub-space is from the 109th row to the 148th row, and the second sub-space is from the 1st row to the 108th row and from the 149th row to the 256th row as shown in FIG. 4B.

The K-space can refer to a frequency space that is a conjugate space of an image space under a Fourier transform. The K-space may be applied to an imaging analysis of magnetic resonance angiography. The above K-spaces in FIG. 4A and FIG. 4B are only examples for convenience of description. The size of the K-space is known in a practical application.

As shown in FIG. 4A, after the size of the third K-space is known, the third echo signal set corresponding to the third K-space may be obtained by adjusting spatial phase encoding. As shown in FIG. 4B, after the size of the third K-space and the central phase encoding line of the third K-space are known, the first sub-space may be firstly determined, and the first sub-space is located near the center of the third K-space. For example, the index of the central phase encoding line in the third K-space is 128.5, and the absolute of the difference between the index of each of the phase encoding lines in the first sub-space and the index of the central phase encoding line in the third K-space is less than the first threshold, for example, 20. Thus, the indexes of the phase encoding lines in the first sub-space are from 109 to 148. Then, the first sub-space echo signals of the third K-space may be collected by adjusting spatial phase encoding, that is, the first sub-space echo signals with the phase encoding indexes being 109-148 are collected.

In the manners for obtaining the third echo signal within the third time interval in each of the second repeated collecting periods to form the third echo signal set, the first manner and the second manner are different as follows: all third echo signals of the third K-space are collected in the first manner, but the first sub-space echo signals of the third K-space are collected in the second manner. That is, the number of the echo signals collected in the second manner is less than the number of the echo signals collected in the first manner, which is not described herein.

Selecting the second sub-space echo signal set corresponding to the second sub-space from the first echo signal set may include: dividing the first K-space corresponding to the first time interval into a third sub-space and a fourth sub-space in a manner same as the first sub-space and the second sub-space are divided. Then, a fourth sub-space echo signal set corresponding to the fourth sub-space is selected from the first echo signal set as the second sub-space echo signal set corresponding to the second sub-space.

For example, as shown in FIG. 4B, in this case, the third K-space shown in FIG. 4B indicates the first K-space. The 1st first echo signal (i.e., the 1st row) in the first echo signal set may be taken as the 1st third echo signal in the third echo signal set, and so on . . . , the 30th first echo signal in the first echo signal set may be taken as the 30th third echo signal in the third echo signal set, and the 72nd first echo signal in the first echo signal set may be taken as the 72nd third echo signal in the third echo signal set, and so on.

Through the above processing, the first sub-space echo signal set and the second sub-space echo signal set are obtained, and then combined to form the third echo signal set.

In the second manner, since the time for obtaining the first echo signal set is approximate to the time for obtaining the third echo signal set, and the signals of the flowing tissue of the subject may not take great change within the two intervals that are spaced closely. Therefore, the slice selecting gradient, the encoding gradient and the readout gradient corresponding to each of the first echo signals in the first echo signal set are same as those corresponding to each of the third echo signals in the third echo signal set. That is, the first echo signal set may be substantially the same as the third echo signal set. Based on this, the second sub-space echo signal set may be directly selected from the first echo signal set, without collecting the second sub-space echo signals by adjusting spatial phase encoding. In this way, the time for obtaining the third echo signal set can be reduced greatly.

In some cases, obtaining the fourth echo signals within the fourth time intervals included in the second repeated collecting periods to form the fourth echo signal set may be performed in any one of a manner A and a manner B.

In the manner A, the fourth echo signal within a fourth K-space corresponding to the fourth time interval in each of the second repeated collecting periods is collected to form the fourth echo signal set. The fourth echo signal set includes the fourth echo signals collected within the second repeated collecting periods, and each of the fourth echo signals corresponds to a respective spatial phase code. The fourth echo signal set may be obtained in a manner similar to that of the first echo signal set, which is not described herein.

In the manner B, the fourth K-space corresponding to the fourth time interval is divided into a fifth sub-space and a sixth sub-space, both of which form the fourth K-space; a fifth sub-space echo signal within the fifth sub-space in each of the second repeated collecting periods is collected to form a fifth sub-space echo signal set without collecting sixth sub-space echo signals in a sixth sub-space echo signal set; the sixth sub-space echo signal set is obtained with a zero filling strategy, that is, each of the sixth sub-space echo signals in the sixth sub-space echo signal set is 0; and the fourth echo signal set is formed by combining the fifth sub-space echo signal set with zero signals in the sixth sub-space echo signal set. It is noted that the fifth sub-space echo signal has been flow-dephased.

When the fourth K-space corresponding to the fourth time interval is divided into the fifth sub-space and the sixth sub-space, an absolute of a difference between an index of each of phase encoding lines in the fifth sub-space and an index of a central phase encoding line in the fourth K-space is less than a second threshold (which may be configured to be, for example, 20, based on experiences), and an absolute of a difference between an index of each of phase encoding lines in the sixth sub-space and the index of the central phase encoding line in the fourth K-space is greater than or equal to the second threshold, and the fifth sub-space and the sixth sub-space can be completely equivalent to the fourth K-space.

In the manners for obtaining the fourth echo signal within the fourth time interval included in each of the second repeated collecting periods to form the fourth echo signal set, the manner A and the manner B are different as follows: all fourth echo signals of the fourth K-space are collected in the manner A, but the fifth sub-space echo signals of the fourth K-space are collected in the manner B, which is not described herein.

Obtaining the sixth sub-space echo signal set with the zero filling strategy may include: filling each of the sixth sub-space echo signals in the sixth sub-space echo signal set with 0, that is, all sixth sub-space echo signals in the sixth sub-space echo signal set are 0.

Through the above processing, the fifth sub-space echo signal set and the sixth sub-space echo signal set may be obtained, and then combined to form the fourth echo signal set.

In the manner B, the sixth sub-space echo signals in the sixth sub-space echo signal set may be obtained by performing zero filling strategy on the sixth sub-space echo signals rather than adjusting the spatial phase encoding, thereby significantly reducing the time for obtaining the sixth sub-space echo signal set.

Since the fifth sub-space echo signal set (i.e., information of the K-space center) in the fourth echo signal set are already collected and main information of a reconstructed image is located in the collected phase encoding lines for the fourth K-space, the collected phase encoding lines for the fourth K-space being located in the center of the fourth K-space, the sixth sub-space echo signals is filled with 0 without causing the loss of the main information, that is, the fourth echo signal set is accurate.

At step 303, a venous blood vessel image, i.e. an image of a venous blood vessel, is reconstructed according to the second echo signal set.

Since each of the second echo signals in the second echo signal set is flow-compensated and the second echo time is relatively longer than the first echo time, the second echo signals in the second echo signal set may highlight the venous blood vessel with a relatively strong magnetic susceptibility effect. Based on this, the venous blood vessel image may be reconstructed with Susceptibility Weighted Imaging (SWI) technology, which is not limited herein.

In the above venous blood vessel image, the venous blood vessel with rich paramagnetic deoxyhemoglobin is highlighted, which may be used for contrast-enhanced display of some diseases, such as, iron deposition in the brain, acute brain injury and so on.

At step 304, an arteriovenous blood vessel image is obtained according to the first echo signal set, the third echo signal set and the fourth echo signal set. The arteriovenous blood vessel image is an image including an arterial blood vessel and a venous blood vessel. A brain parenchymal background signal in the arteriovenous blood vessel image may be basically removed through reconstruction processing.

In some cases, obtaining the arteriovenous blood vessel image according to the first echo signal set, the third echo signal set and the fourth echo signal set includes: obtaining a first sub-image according to the first echo signal set; obtaining a second sub-image according to the third echo signal set; obtaining a third sub-image according to the first sub-image and/or the second sub-image; obtaining a fourth sub-image according to the fourth echo signal set; and obtaining the arteriovenous blood vessel image according to the third sub-image and the fourth sub-image.

Further, obtaining the third sub-image according to the first sub-image and/or the second sub-image may include one of the followings: determining the first sub-image as the third sub-image; determining the second sub-image as the third sub-image; and obtaining the third sub-image by performing averaging processing on the first sub-image and the second sub-image. If the third sub-image is obtained by performing averaging processing on the first sub-image and the second sub-image, that is, by performing averaging processing (accumulation processing) on the two sub-images, a signal-to-noise ratio of the third sub-image can be increased and a blood tissue signal can be enhanced.

Further, obtaining the arteriovenous blood vessel image according to the third sub-image and the fourth sub-image may include, but not limited to: obtaining the arteriovenous blood vessel image by performing subtracting processing on the third sub-image and the fourth sub-image.

In an example, since each of the first echo signals in the first echo signal set is flow-compensated and the flow compensation has a phase convergence effect, information of the arterial blood vessel and the venous blood vessel is retained. Therefore, the first echo signal set includes information of the arterial blood vessel, the venous blood vessel and a background tissue (an organ other than the arterial blood vessel and the venous blood vessel). Similarly, the third echo signal set may also include the information of the arterial blood vessel, the venous blood vessel and the background tissue. In conclusion, the third sub-image may include the information of the arterial blood vessel, the venous blood vessel and the background tissue.

Since each of the fourth echo signals in the fourth echo signal set is flow-dephased and the flow dephasing has a phase divergence effect, the information in which the blood tissue signals for the arterial blood vessel and the venous blood vessel are weakened is obtained. Therefore, the fourth echo signal set may include the information in which the blood tissue signals are weakened but the background tissue is basically unaffected. In conclusion, the fourth sub-image may include the information in which the background tissue is basically unaffected and the blood tissue signals for the arterial blood vessel and the venous blood vessel are weakened. Based on this, when the subtracting processing is performed on the third sub-image and the fourth sub-image, the arteriovenous blood vessel image without the background tissue may be obtained. That is, the arteriovenous blood vessel image includes the information of the arterial blood vessel and the venous blood vessel, and in the arteriovenous blood vessel image, the background tissue is suppressed. In this way, in the arteriovenous blood vessel image, the background tissue is eliminated, and the blood tissue signal is highlighted.

In an example, it is assumed that the third sub-image is S_{RP}, S_{RP}=S_{A}+S_{B}, S_{A} refers to information of the arterial blood vessel and the venous blood vessel, and S_{B} refers to information of the background tissue; and it is assumed that the fourth sub-image is S_{DP}, S_{DP}=S_{A}′+S_{B}′, S_{A}′ refers to information of the arterial blood vessel and the venous blood vessel, and S_{B}′ refers to information of the background tissue. Since the background tissue is basically unaffected when the flow compensation or flow dephasing is performed, S_{B} and S_{B}′ are approximately the same, that is, S_{B} is approximately equal to S_{B}′. Descriptions are made below with S_{B} being equal to S_{B}′. Since the information of the arterial blood vessel and the venous blood vessel can be retained when the flow compensation is performed, and the blood tissue signal corresponding to the flow speed can be weakened when the flow dephasing is performed, S_{A} is far greater than S_{A}′, S_{A}′ is approximate to 0, and S_{A} is the actual information of the arterial blood vessel and the venous blood vessel.

In conclusion, when the subtracting processing is performed on the third sub-image and the fourth sub-image, the obtained vessel signal is a difference between S_{A} and S_{A}′, that is, the obtained vessel signal corresponds to the arteriovenous blood vessel image without the background tissue, thereby enhancing a contrast between the arteriovenous blood vessel and the background tissue.

At step 305, an arterial blood vessel image is obtained according to the venous blood vessel image and the arteriovenous blood vessel image. The arterial blood vessel image may be obtained by performing the subtracting processing on the arteriovenous blood vessel image and the venous blood vessel image.

The above venous blood vessel image is an image including a venous blood vessel and the above arteriovenous blood vessel image is an image including an arterial blood vessel and a venous blood vessel. Therefore, an arterial blood vessel image including an arterial blood vessel may be obtained by performing the subtracting processing on the arteriovenous blood vessel image and the venous blood vessel image.

In the above examples, for the process of obtaining an image according to echo signals, the radiofrequency receiving coil 130 is configured to transmit the echo signals to a spectrometer, the spectrometer is configured to convert the echo signals into image signals and then transmit the image signals to a computer which may then generate an image by using the image signals. For example, the radiofrequency receiving coil 130 may transmit the second echo signal set to the spectrometer, the spectrometer may convert the second echo signal set into image signals and transmit the image signals to the computer, and the computer may reconstruct the venous blood vessel image by using the image signals.

Based on the above technical solution, the arterial blood vessel image (a high-contrast image of an artery and a background tissue) and the venous blood vessel image (a high-contrast image of a vein and a background tissue) may be provided to implement high contrast imaging of entire blood vessel tissues, thereby improving user experience and increasing application value. The magnetic resonance angiography method provided by the present disclosure can bring an important value to cerebral neuroscience study and clinical application. For example, the magnetic resonance angiography method provided by the present disclosure is very important for the auxiliary diagnosis of diseases, such as cerebral arteriovenous malformation, stroke, acute brain injury, tumours and so on.

FIG. 5 is a schematic diagram illustrating a hardware structure of a magnetic resonance angiography apparatus according to one or more examples of the present disclosure. The magnetic resonance angiography apparatus may be applied in an MRI device. The MRI device may include the various components shown in FIG. 1 and the magnetic resonance angiography apparatus. The magnetic resonance angiography apparatus includes a processor 510, a communication interface 520, a non-transitory machine readable storage medium 530 and an internal bus 540. The processor 510, the communication interface 520 and the non-transitory machine readable storage medium 530 are typically connected to each other by the internal bus 540.

The processor 510 may read the machine executable instructions stored in the non-transitory machine readable storage medium 530 to perform the magnetic resonance angiography method in the above examples of the present disclosure.

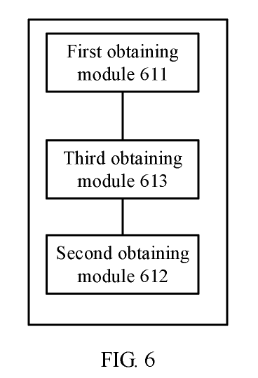

The non-transitory machine readable storage medium 530 stores machine readable instructions corresponding to the magnetic resonance angiography logic. FIG. 6 is a schematic diagram illustrating a structure of a magnetic resonance angiography logic according to one or more examples of the present disclosure. The magnetic resonance angiography logic includes a first obtaining module 611, a second obtaining module 612 and a third obtaining module 613.

The first obtaining module 611 is configured to, in each of a plurality of first repeated collecting periods, obtain a first echo signal within a first time interval included in the first repeated collecting period to form a first echo signal set and obtain a second echo signal within a second time interval included in the first repeated collecting period to form a second echo signal set. The first echo signal has been flow-compensated, and the second echo signal has been flow-compensated.

The second obtaining module 612 is configured to, in each of the plurality of second repeated collecting periods, obtain a third echo signal within a third time interval included in the second repeated collecting period to form a third echo signal set and obtain a fourth echo signal within a fourth time interval included in the second collecting period to form a fourth echo signal set. The third echo signal has been flow-compensated. The fourth echo signal has been flow-dephased.

The third obtaining module 613 is configured to reconstruct a venous blood vessel image according to the second echo signal set, obtain an arteriovenous blood vessel image according to the first echo signal set, the third echo signal set and the fourth echo signal set, and obtain an arterial blood vessel image according to the venous blood vessel image and the arteriovenous blood vessel image.

For convenience of description, the magnetic resonance angiography logic is divided into different modules based on functions for descriptions. Of course, the functions of different modules may be implemented in one or more softwares and/or hardwares. It is noted that each of the modules corresponds to the magnetic resonance angiography method. Further detail is omitted for brevity.

The persons skilled in the art should understand that the examples of the present disclosure may be provided as a method, a system, or a computer program product. Thus, entire hardware examples, entire software examples or examples combining software and hardware may be applied in examples of the present disclosure. Further, the present disclosure may be implemented in the form of a computer program product that is operated on one or more computer available storage media (including but not limited to magnetic disk memory, CD-ROM, and optical memory and so on) including computer available program codes.

The present disclosure is described by referring to flowcharts and/or block diagrams of a method, a device (a system) and a computer program product in examples of the present disclosure. It is understood that each flowchart and/or block in the flowcharts and/or the block diagrams or a combination of a flow chart and/or a block of the flowcharts and/or the block diagrams may be implemented by computer program instructions. These computer program instructions may be provided to a general-purpose computer, a dedicated computer, an embedded processor, or a processor of another programmable data processing device to generate a machine, so that the instructions executable by a computer or a processor of another programmable data processing device generate an apparatus for implementing functions designated in one or more flows of the flowcharts and/or one or more blocks of the block diagrams.

Further, these computer program instructions may also be stored in a computer readable memory that can direct a computer or another programmable data processing device to work in a particular manner, so that the instructions stored in the computer readable memory generate a product including an instruction apparatus and the instruction apparatus can implement functions designated in one or more flows of the flowcharts and/or one or more blocks of the block diagrams.

These computer program instructions may also be loaded on a computer or another programmable data processing device, so that a series of operation blocks can be executed on the computer or another programmable device to generate processing achieved by the computer, and thus instructions executable on the computer or another programmable data processing device are provided for blocks for implementing functions designated in one or more flows of the flowcharts and/or one or more blocks of the block diagrams.

The term used in the present disclosure is for the purpose of describing a particular example only, and is not intended to limit the present disclosure. The singular forms such as “a”, ‘said”, and “the” used in the present disclosure and the appended claims are also intended to include multiple, unless the context clearly indicates otherwise. It is also to be understood that the term “and/or” as used herein refers to any or all possible combinations that include one or more associated listed items.

It is to be understood that although different information may be described using the terms such as first, second, third, etc. in the present disclosure, the information should not be limited to these terms. These terms are used only to distinguish the same type of information from each other. For example, without departing from the scope of the present disclosure, the first information may also be referred to as the second information, and similarly, the second information may also be referred to as the first information. Depending on the context, the word “if” as used herein may be interpreted as “when” or “as” or “determining in response to”.

The foregoing descriptions are only examples of the present disclosure but not intended to limit the present disclosure. For those skilled in the art, various modifications and changes may be made to the present disclosure. Any modifications, equivalent substitutions, and improvements made within the spirit and principles of the disclosure shall be encompassed in the scope of protection of the present disclosure.

What is claimed is:

1. A magnetic resonance angiography method comprising:
forming a first echo signal set in a plurality of first repeated collecting periods by collecting a respective first echo signal within a first time interval included in each of the first repeated collecting periods, wherein the respective first echo signals have been flow-compensated;
forming a second echo signal set in the plurality of first repeated collecting periods by collecting a respective second echo signal within a second time interval included in each of the first repeated collecting periods, wherein the respective second echo signals have been flow-compensated;
forming a third echo signal set in a plurality of second repeated collecting periods by obtaining third echo signals within third time intervals included in the plurality of second repeated collecting periods, wherein the third echo signals have been flow-compensated;
forming a fourth echo signal set in the plurality of second repeated collecting periods by obtaining fourth echo signals within fourth time intervals included in the plurality of second repeated collecting periods, wherein the fourth echo signals have been flow-dephased;
reconstructing a venous blood vessel image according to the second echo signal set;
obtaining an arteriovenous blood vessel image according to the first echo signal set, the third echo signal set and the fourth echo signal set; and
obtaining an arterial blood vessel image according to the venous blood vessel image and the arteriovenous blood vessel image.

2. The method of claim 1, wherein a first echo time for the first time interval is the same as a third echo time for the third time interval,
wherein a second echo time for the second time interval is the same as a fourth echo time for the fourth time interval, and
wherein the second echo time is longer than the first echo time, and the fourth echo time is longer than the third echo time.

3. The method of claim 1, wherein forming a third echo signal set in a plurality of second repeated collecting periods comprises:
collecting a respective third echo signal within a third time interval included in each of the second repeated collecting periods.

4. The method of claim 1, wherein forming a third echo signal set in a plurality of second repeated collecting periods comprises:
dividing a third K-space corresponding to the third time intervals into a first sub-space and a second sub-space, wherein an absolute value of a difference between an index of each of phase encoding lines in the first sub-space and an index of a central phase encoding line in the third K-space is less than a first threshold, and an absolute value of a difference between an index of each of phase encoding lines in the second sub-space and the index of the central phase encoding line in the third K-space is greater than or equal to the first threshold;
collecting a first sub-space echo signal within the first sub-space in the third time interval included in each of the second repeated collecting periods to form a first sub-space echo signal set, wherein the first sub-space echo signals have been flow-compensated;
selecting a second sub-space echo signal set corresponding to the second sub-space from the first echo signal set; and
forming the third echo signal set by combining the first sub-space echo signal set and the second sub-space echo signal set.

5. The method of claim 4, wherein selecting the second sub-space echo signal set corresponding to the second sub-space from the first echo signal set comprises:
dividing a first K-space corresponding to the first time intervals into a third sub-space and a fourth sub-space, wherein the first echo signals of the first echo signal set are filled in rows of the first K-space, and wherein an absolute value of a difference between an index of each of phase encoding lines in the third sub-space and an index of a central phase encoding line in the first K-space is less than the first threshold, and an absolute of a difference between an index of each of phase encoding lines in the fourth sub-space and the index of the central phase encoding line in the first K-space is greater than or equal to the first threshold; and
selecting a fourth sub-space echo signal set corresponding to the fourth sub-space from the first echo signal set as the second sub-space echo signal set corresponding to the second sub-space.

6. The method of claim 1, wherein forming a fourth echo signal set in the plurality of second repeated collecting periods comprises:
collecting a respective fourth echo signal within a fourth time interval included in each of the second repeated collecting periods.

7. The method of claim 1, wherein forming a fourth echo signal set in the plurality of second repeated collecting periods comprises:
dividing a fourth K-space corresponding to the fourth time interval into a fifth sub-space and a sixth sub-space, wherein an absolute of a difference between an index of each of phase encoding lines in the fifth sub-space and an index of a central phase encoding line in the fourth K-space is less than a second threshold, and an absolute of a difference between an index of each of phase encoding lines in the sixth sub-space and the index of the central phase encoding line in the fourth K-space is greater than or equal to the second threshold;
collecting a fifth sub-space echo signal within the fifth sub-space in each of the second repeated collecting periods to form a fifth sub-space echo signal set, wherein the fifth sub-space echo signals have been flow-dephased;
obtaining a sixth sub-space echo signal set with a zero filling strategy; and
forming the fourth echo signal set by combining the fifth sub-space echo signal set and the sixth sub-space echo signal set.

8. The method of claim 1, wherein obtaining the arteriovenous blood vessel image according to the first echo signal set, the third echo signal set and the fourth echo signal set comprises:
obtaining a first sub-image according to the first echo signal set;
obtaining a second sub-image according to the third echo signal set;
obtaining a third sub-image according to at least one of the first sub-image and the second sub-image;
obtaining a fourth sub-image according to the fourth echo signal set; and
obtaining the arteriovenous blood vessel image according to the third sub-image and the fourth sub-image.

9. The method of claim 8, wherein obtaining the third sub-image according to at least one of the first sub-image and the second sub-image comprises one of the following:
determining the first sub-image as the third sub-image;
determining the second sub-image as the third sub-image; and
obtaining the third sub-image by performing averaging processing on the first sub-image and the second sub-image.

10. The method of claim 8, wherein obtaining the arteriovenous blood vessel image according to the third sub-image and the fourth sub-image comprises:
obtaining the arteriovenous blood vessel image by performing subtracting processing on the third sub-image and the fourth sub-image.

11. The method of claim 1, wherein obtaining the arterial blood vessel image according to the venous blood vessel image and the arteriovenous blood vessel image comprises:
obtaining the arterial blood vessel image by performing subtracting processing on the arteriovenous blood vessel image and the venous blood vessel image.

12. A magnetic resonance angiography apparatus, comprising:
at least one processor; and
at least one non-transitory machine-readable storage medium coupled to the at least one processor having machine-executable instructions stored thereon that, when executed by the at least one processor, cause the at least one processor to perform operations comprising:
forming a first echo signal set in a plurality of first repeated collecting periods by collecting a respective first echo signal within a first time interval included in each of the first repeated collecting periods, wherein the respective first echo signals have been flow-compensated;
forming a second echo signal set in the plurality of first repeated collecting periods by collecting a respective second echo signal within a second time interval included in each of the first repeated collecting periods, wherein the respective second echo signals have been flow-compensated;
forming a third echo signal set in a plurality of second repeated collecting periods by obtaining third echo signals within third time intervals included in the plurality of second repeated collecting periods, wherein the third echo signals have been flow-compensated;
forming a fourth echo signal set in the plurality of second repeated collecting periods by obtaining fourth echo signals within fourth time intervals included in the plurality of second repeated collecting periods, wherein the fourth echo signals have been flow-dephased;
reconstructing a venous blood vessel image according to the second echo signal set;
obtaining an arteriovenous blood vessel image according to the first echo signal set, the third echo signal set and the fourth echo signal set; and
obtaining an arterial blood vessel image according to the venous blood vessel image and the arteriovenous blood vessel image.

13. The magnetic resonance angiography apparatus of claim 12, wherein a first echo time for the first time interval is the same as a third echo time for a third time interval,
wherein a second echo time for the second time interval is the same as a fourth echo time for a fourth time interval, and
wherein the second echo time is longer than the first echo time, and the fourth echo time is longer than the third echo time.

14. The magnetic resonance angiography apparatus of claim 12, wherein forming a third echo signal set in a plurality of second repeated collecting periods comprises:
collecting a respective third echo signal within a third time interval included in each of the second repeated collecting periods.

15. The magnetic resonance angiography apparatus of claim 12, wherein forming a third echo signal set in a plurality of second repeated collecting periods comprises:
dividing a third K-space corresponding to the third time intervals into a first sub-space and a second sub-space, wherein an absolute value of a difference between an index of each of phase encoding lines in the first sub-space and an index of a central phase encoding line in the third K-space is less than a first threshold, and an absolute value of a difference between an index of each of phase encoding lines in the second sub-space and the index of the central phase encoding line in the third K-space is greater than or equal to the first threshold;
collecting a first sub-space echo signal within the first sub-space in the third time interval included in each of the second repeated collecting periods to form a first sub-space echo signal set, wherein the first sub-space echo signals have been flow-compensated;
selecting a second sub-space echo signal set corresponding to the second sub-space from the first echo signal set; and
forming the third echo signal set by combining the first sub-space echo signal set and the second sub-space echo signal set.

16. The magnetic resonance angiography apparatus of claim 15, wherein selecting the second sub-space echo signal set corresponding to the second sub-space from the first echo signal set comprises:
dividing a first K-space corresponding to the first time intervals into a third sub-space and a fourth sub-space, wherein the first echo signals of the first echo signal set are filled in rows of the first K-space, and wherein an absolute value of a difference between an index of each of phase encoding lines in the third sub-space and an index of a central phase encoding line in the first K-space is less than the first threshold, and an absolute of a difference between an index of each of phase encoding lines in the fourth sub-space and the index of the central phase encoding line in the first K-space is greater than or equal to the first threshold; and
selecting a fourth sub-space echo signal set corresponding to the fourth sub-space from the first echo signal set as the second sub-space echo signal set corresponding to the second sub-space.

17. The magnetic resonance angiography apparatus of claim 12, wherein forming a fourth echo signal set in the plurality of second repeated collecting periods comprises:
collecting a respective fourth echo signal within a fourth time interval included in each of the second repeated collecting periods.

18. The magnetic resonance angiography apparatus of claim 12, wherein forming a fourth echo signal set in the plurality of second repeated collecting periods comprises:
dividing a fourth K-space corresponding to the fourth time interval into a fifth sub-space and a sixth sub-space, wherein an absolute of a difference between an index of each of phase encoding lines in the fifth sub-space and an index of a central phase encoding line in the fourth K-space is less than a second threshold, and an absolute of a difference between an index of each of phase encoding lines in the sixth sub-space and the index of the central phase encoding line in the fourth K-space is greater than or equal to the second threshold;
collecting a fifth sub-space echo signal within the fifth sub-space in each of the second repeated collecting periods to form a fifth sub-space echo signal set, wherein the fifth sub-space echo signals have been flow-dephased;
obtaining a sixth sub-space echo signal set with a zero filling strategy; and
forming the fourth echo signal set by combining the fifth sub-space echo signal set and the sixth sub-space echo signal set.

19. The magnetic resonance angiography apparatus of claim 12, wherein obtaining the arteriovenous blood vessel image according to the first echo signal set, the third echo signal set and the fourth echo signal set comprises:
obtaining a first sub-image according to the first echo signal set;
obtaining a second sub-image according to the third echo signal set;
obtaining a third sub-image according to at least one of the first sub-image and the second sub-image;
obtaining a fourth sub-image according to the fourth echo signal set; and
obtaining the arteriovenous blood vessel image according to the third sub-image and the fourth sub-image.

20. The magnetic resonance angiography apparatus of claim 12, wherein obtaining the arterial blood vessel image according to the venous blood vessel image and the arteriovenous blood vessel image comprises:
obtaining the arterial blood vessel image by performing subtracting processing on the arteriovenous blood vessel image and the venous blood vessel image.
